(19) United States
(12) Patent Application Publication
(10) Pub. No.: US 2007/0179147 A1
(43) Pub. Date: Aug. 2, 2007

(54) Methods of treating and preventing Alzheimer's disease

(76) Inventors: Zhi-Gang Jiang, Gaithersburg, MD (US); Hossein A. Ghanbari, Potomac, MD (US)

Correspondence Address: PANACEA PHARMACEUTICALS, INC., 207 PERRY PARKWAY, SUITE 2, GAITHERSBURG, MD, 20877, US

(21) Appl. No.: 11/340,927

(22) Filed: Jan. 27, 2006

(51) Int. Cl. A61K 31/4965; A61K 31/44; A61K 31/426; A61K 31/4172

(52) U.S. Cl. 514/252.1; 514/357; 514/365; 514/400; 514/352

(57) ABSTRACT

The present invention relates to methods of treating or preventing the progression of Alzheimer's disease, by administering to a patient in need thereof certain thiosemicarbazone compounds. More particularly, the present invention relates to methods of preventing or treating neuronal damage and neuronal cell death occurring as a result of cellular insult of an amyloid-beta peptide. An example of such a thiosemicarbazone is 3-aminopyridine-2-carboxaldehyde thiosemicarbazone (PAN-811).

### FIELD OF THE INVENTION

The present invention relates to methods of treating or preventing the progression of Alzheimer's disease, by administering to a patient in need thereof certain thiosemicarbazone compounds. More particularly, the present invention relates to methods of preventing or treating neuronal damage and cell death occurring as a result of cellular insult by an amyloid-beta peptide. The methods involve the use of certain thiosemicarbazone compounds.

### BACKGROUND OF THE INVENTION

The present invention is broadly directed to a new use of certain N-heterocyclic carboxaldehyde thiosemicarbazones (HCTs), which have up to now been known as useful as antineoplastic agents, acting as potent inhibitors of ribonucleotide reductase. Methods of treatment of tumors using such compounds are disclosed inter alia in U.S. Pat. Nos. 5,721,259 and 5,281,715 of Sartorelli et al.

More recently, U.S. Pat. No. 6,613,803 disclosed the use of certain novel thiosemicarbazones for the treatment of neuronal damage and neurodegenerative diseases. The novel compounds are described as exerting their therapeutic effects as sodium channel blockers.

However, until now there has been no disclosure in the art of the use of compounds that are the same or similar to those disclosed in the Sartorelli patents for treating or preventing neuronal damage in Alzheimer's disease.

While no therapy for neuroprotection is currently marketed, there are drugs approved for use in the therapy of chronic neurological conditions, which are glutamate receptor (NMDA) antagonists. Although there is evidence of ameliorating affects of such drugs in chronic CNS degenerative states, it does not appear that NMDA antagonists, alone, can provide substantial protection against neuronal insult by amyloid-beta.

Multiple mechanisms by which amyloid-beta contribute to neuronal cell death have been proposed in the literature. While the precise mechanism is not fully understood, previous studies have shown that amyloid-beta (“Aβ1-42”) leads to neuronal insult and death both in vivo and in vitro.

The development of therapeutic agents effective in preventing or ameliorating neuron damage and its consequences in Alzheimer's disease, in particular that damage caused by the actions of Aβ1-42, is highly desirable.

### SUMMARY OF THE INVENTION

The present invention relates to methods of treating or preventing the progression of Alzheimer's disease, by administering to a patient in need thereof certain N-heterocyclic 2-carboxaldehyde thiosemicarbazones (HCTs) and pharmaceutically acceptable salts or prodrugs thereof: Such useful compounds are embraced by Formula I:

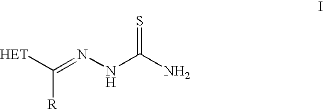

More preferably, the compound is selected from a compound of Formula II-VI, infra.

As a most preferred embodiment, PAN-811 (3-aminopyridine-2-carboxaldehyde thiosemicarbazone) is used to practice the methods of the present invention, which has the formula:

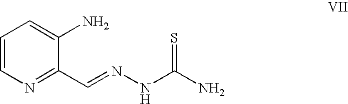

### BRIEF DESCRIPTION OF THE DRAWINGS

FIG. 1 contains graphic representations of cell viability (left panel) and neuroprotective capacity (right panel) after pre-treatment with PAN-811 (A) or known neuroprotectants Vitamin E (B), lipoic acid (C), or Ginkgo biloba (D) and subsequent treatment with H₂O₂.

FIG. 2 contains graphic representations of the effects of PAN-811 on ROS generation in neuronal cells. (A); the effects of PAN-811 on H₂O₂-induced ROS generation in neuronal cells. (B); the effects of PAN-811 on the basal level of ROS generation in neuronal cells.

FIG. 3 is a graphic representation of the dependence of neurotoxicity on the concentration of glucose in hypoxic conditions.

FIG. 4 shows representative histological photographs of cells under hypoxic conditions with and without neuroprotectants, MK801 and PAN-811.

FIG. 5 is a graphic representation of the neuroprotective effects of PAN-811 under normoxic and hypoxic conditions.

FIG. 6 depicts graphic representations of the toxicity of PAN-811, under hypoxic/hypoglycemic conditions.

FIG. 7 is a graphic representation of the protective effects of PAN-811 on neuronal cell death due to mild hypoxic/hypoglycemic conditions.

FIG. 8 is a graphic representation of the neurotoxicity of PAN-811 where cortical neurons were treated with PAN-811 for 24 hours.

FIG. 9 is a graphic representation of the protective effects of PAN-811 against toxicity due to ischemia.

FIG. 10 shows graphic representations of cell viability after pre-treatment with PAN-811 or solvent and treatment with H₂O₂.

FIG. 11 shows graphic representations of cell viability after pre-treatment with PAN-811 or solvent and treatment with H₂O₂.

### DETAILED DESCRIPTION OF THE INVENTION

The present invention relates to methods of treating and preventing neuronal damage in Alzheimer's disease subjects, by administering to a patient in need of such treatment a compound of Formula I, or pharmaceutically acceptable salts or prodrugs thereof: 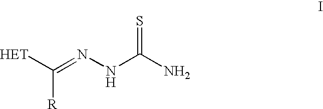
where HET is a 5 or 6 membered heteroaryl residue having 1 or 2 heteroatoms selected from N and S, and optionally substituted with an amino group; and R is H or C₁-C₄- alkyl.

In one preferred embodiment, the compound is of Formula II: 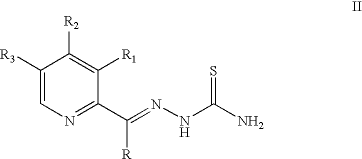
where R is H or C₁-C₄- alkyl; and R₁, R₂ and R₃ are independently selected from H and amino.

In another preferred embodiment, the compound is of Formula III: 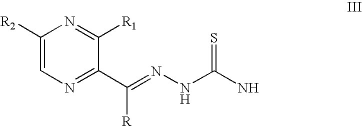
where R is H or C₁-C₄- alkyl; and R₁ and R₂ are independently selected from H and amino.

In another preferred embodiment, the compound is of Formula IV: 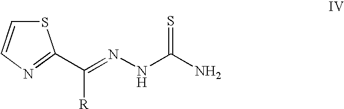
where R is H or C₁-C₄- alkyl.

Yet another preferred embodiment is a compound of formula V: 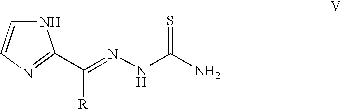
where R is R is H or C₁-C₄- alkyl.

Finally, another preferred embodiment is a compound of Formula VI: 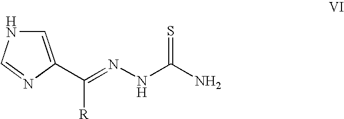
where R is H or C₁-C₄- alkyl.

As more preferred embodiments, the compounds of the present invention are selected from:
(1) Formula II, where R is methyl, and R₁, R₂ and R₃ are H;
(2) Formula III, where R is methyl and R₁ and R₂ are H;
(3) Formula IV, where R is methyl;
(4) Formula IV, where R is H;
(5) Formula V, where R is H; and
(6) Formula VI, where R is H.

A most preferred embodiment of the present invention relates to methods of treating or preventing the progression of Alzheimer's disease, by administering to a patient in need of such treatment 3-aminopyridine-2-carboxaldehyde thiosemicarbazone (“PAN-811”), which has the formula:

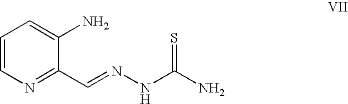

Certain of the compounds of the present invention may exist as E, Z-stereoisomers about the C═N double bond and the invention includes the mixture of isomers as well as the individual isomers that may be separated according to methods that are well known to those of ordinary skill in the art. Certain of the compounds of the present invention may exist as optical isomers and the invention includes both the racemic mixtures of such optical isomers as well as the individual entantiomers that may be separated according to methods that are well known to those of ordinary skill in the art.

Examples of pharmaceutically acceptable salts are inorganic and organic acid addition salts such as hydrochloride, hydrobromide, phosphate, sulphate, citrate, lactate, tartrate, maleate, fumarate, acetic acid, dichloroacetic acid and oxalate.

Examples of prodrugs include, for instance, esters of the compounds with R₁-R₃ as hydroxyalkyl, and these may be prepared in accordance with known techniques.

It is surprising and unexpected that the compound, 3-aminopyridine-2-carboxaldehyde thiosemicarbazone, and several new analogs thereof, are effective as neuroprotectants against the cellular insult of amyloid-beta, given that its only publicly known use thus far has been as an antineoplastic agent. See, for example, U.S. Pat. No. 5,721,259.

Thus, one of the embodiments of the present invention is directed to the amelioration of the effects of amyloid-beta on nerve cells and tissue and, particularly, preventing neuronal damage by amyloid-beta at any stage of Alzheimer's disease. The present invention also contemplates the prophylactic administration of the compounds in subjects suspected of a familial or genetic risk for developing Alzheimer's disease.

The means for synthesizing compounds useful in the methods of the invention are well known in the art. Such synthetic schemes are described in U.S. Pat. Nos.: 5,281,715; 5,767,134; 4,447,427; 5,869,676; and 5,721,259, all of which are incorporated herein by reference in their entireties.

In another aspect, the invention is directed to pharmaceutical compositions of the 2-caboxyaldehyde thiosemicarbazones useful in the methods of the invention. The pharmaceutical compositions of the invention comprise one or more of the compounds (or one of the compounds together with one or more different active ingredients) and a pharmaceutically acceptable carrier or diluent. As used herein “pharmaceutically acceptable carrier or diluent” includes any and all solvents, dispersion media, solid excipients (e.g., binders, lubricants, etc. typically used in solid oral dosage forms) coatings, antibacterial and antifungal agents, isotonic and absorption delaying agents, and the like that are physiologically compatible. The type of carrier can be selected based upon the intended route of administration.

In various embodiments, the carrier is suitable for intravenous, intraperitoneal, subcutaneous, intramuscular, topical, transdermal or oral administration. For example, pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. In all dosage forms, supplementary active compounds may be incorporated into the compositions as well.

Preferably, administration is oral, and may be of an immediate or delayed release. Such oral pharmaceutical compositions of the present invention are manufactured by techniques typically used in the pharmaceutical industry. Generally, the active agent(s) is/are preferably formulated into a tablet or capsule for oral administration, prepared using methods known in the art, for instance wet granulation and direct compression methods. The oral tablets are prepared using any suitable process known to the art. See, for example, Remington's Pharmaceutical Sciences, 18^{th} Edition, A. Gennaro, Ed., Mack Pub. Co. (Easton, Pa. 1990), Chapters 88-91, the entirety of which is hereby incorporated by reference. Typically, the active ingredient, i.e., one or more of the thiosemicarbazones, is mixed with pharmaceutically acceptable excipients (e.g., the binders, lubricants, etc.) and compressed into tablets. Preferably, such a dosage form is prepared by a wet granulation technique or a direct compression method to form uniform granulates. Alternatively, the active ingredient(s) can be mixed with a previously prepared non-active granulate. The moist granulated mass is then dried and sized using a suitable screening device to provide a powder, which can then be filled into capsules or compressed into matrix tablets or caplets, as desired.

In one such aspect, the tablets are prepared using a direct compression method. The direct compression method offers a number of potential advantages over a wet granulation method, particularly with respect to the relative ease of manufacture. In the direct compression method, at least one pharmaceutically active agent and the excipients or other ingredients are sieved through a stainless steel screen, such as a 40 mesh steel screen. The sieved materials are then charged to a suitable blender and blended for an appropriate time. The blend is then compressed into tablets on a rotary press using appropriate tooling.

Alternatively, the pharmaceutical composition is contained in a capsule containing beadlets or pellets. Methods for making such pellets are known in the art (see, Remington's, supra). The pellets are filled into capsules, for instance gelatin capsules, by conventional techniques.

Sterile injectable solutions can be prepared by incorporating a desired amount of the active compound in a pharmaceutically acceptable liquid vehicle and filter sterilized. Generally, dispersions may be prepared by incorporating the active compound into a sterile vehicle containing a basic dispersion medium. In the case of sterile powders for the preparation of sterile injectable solutions, the preferred methods of preparation are vacuum drying and freeze-drying, which will yield a powder of the active ingredient plus any additional desired ingredient from a previously sterile-filtered solution thereof.

The pharmaceutical compositions of the present invention may be administered by any means to achieve their intended purpose, for example, by oral, parenteral, subcutaneous, intravenous, intramuscular, intraperitoneal, transdermal, or buccal routes.

The active agent(s) in the pharmaceutical composition (i.e., one or more of the thiosemicarbazones) is present in a therapeutically effective amount. By a “therapeutically effective amount” is meant an amount effective, at dosages and for periods of time necessary, to achieve the desired therapeutic result of positively influencing the course of a particular disease state. This terminology also contemplates and encompasses the therapeutic use of the compounds in a prophylactic manner, which may be of a lower dosage, and such an embodiment is included in the present invention. Of course, therapeutically effective amounts of the active agent(s) may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects.

The amount of active compound in the composition may vary according to factors such as the disease state, age, sex, and weight of the individual. Dosage regimens may be adjusted to provide the optimum therapeutic response. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. It is contemplated that the dosage units of the present invention will contain the active agent(s) in amounts suitable for a dosage regimen of about the same as or, more preferably less than, those presently employed in antineoplastic treatment (e.g., Triapine®, Vion Pharmaceuticals, Inc.). Based on the studies in the Examples, the effective dose of PAN-811 for neuroprotection appears well below its maximal tolerated dose, as well as being below the dosages typically used for cancer treatment. It is contemplated that the disclosed analogues have similar pharmacodynamic profiles as that of PAN-811, and thus the dosage regimens will be the same or similar to PAN-811 for neuroprotection.

The pharmaceutical compositions of the invention may be administered to any animal in need of the beneficial effects of the compounds of the invention. While the invention is primarily directed to human use, other mammals in which an Alzheimer's disease condition is suspected may be treated accordingly if so desired.

This invention is further illustrated by the following examples, which are not intended to limit the present invention. The contents of all references, patents, and published patent applications cited throughout this application are specifically and entirely incorporated herein by reference.

### EXAMPLES

### Example 1

### Comparison of the Neuroprotective Potency of PAN-811 with Other Known Neuroprotectants

The purpose of this study was to compare the neuroprotective capacity of PAN-811 (3-aminopyridine-2-carboxaldehyde thiosemicarbazone; C₇H₉N₅S; MW=195) with known neuroprotectants, such as vitamin E, lipoic acid and Ginkgo biloba, in a cell-based model of Alzheimer's disease-associated oxidative stress.

Isolation and Acculturation of Cells

Primary cortical neurons were isolated from a 17-day old rat embryonic brain and seeded on 96-well plate at 50,000 cells/well in regular neurobasal medium for 2-3 weeks. Twice, half the amount of medium was replaced with fresh neurobasal medium containing no antioxidants.

Treatment with PAN-811, Other Known Neuroprotectants and H₂O₂

PAN-811 was dissolved in EtOH at 1 mg/ml (˜5 mM), and further diluted in medium to final concentration at 0.1 μM, 1 μM, and 10 μM. The other known neuroprotectants were dissolved in appropriate solvents and diluted to the final concentrations as indicated. Neurons were pre-treated with PAN-811, known neuroprotectants, or control vehicle for 24 hours, and then subjected to oxidative stress induced by hydrogen peroxide (final concentration 150 μM). Controls included untreated cells (no compounds and hydrogen peroxide treatment), cells treated with compound only, and cells exposed to hydrogen peroxide but not compounds. Untreated cells were used as a control to evaluate both toxicity and viability of neurons. Each assay was performed in triplicate.

Evaluation of Cellular Function

After 24 hours, the cultures were evaluated for viability and mitochondrial function using a standard MTS Assay (Promega). The manufacturer's protocols were followed.

Materials

Neurobasal medium (Invitrogen); B27-AO, (Invitrogen); PAN-811 (Vion Pharmaceuticals); hydrogen peroxide (Calbiochem); EtOH (Sigma); Vitamin E (Sigma); lipoic acid (Sigma); Ginkgo biloba (CVS); MTS assay kit (Promega)

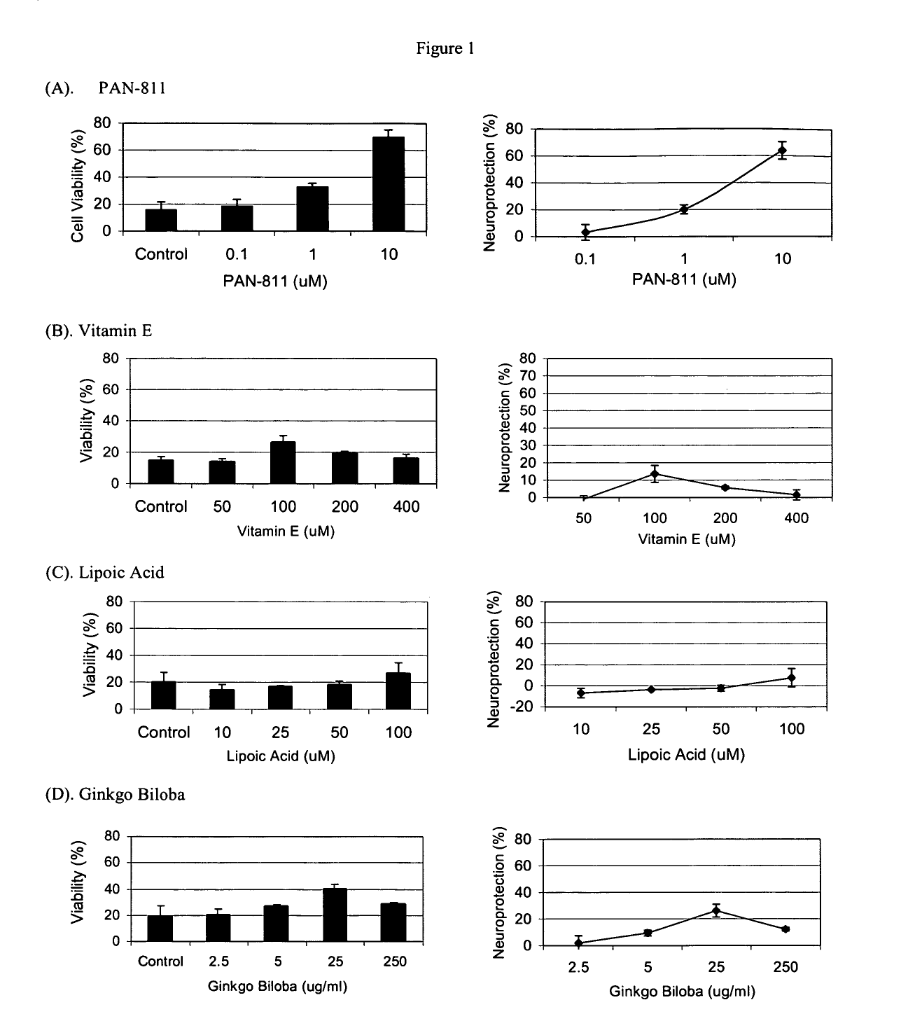

Experiments were carried out in accordance with the above study design. PAN-811 was dissolved in EtOH at 1 mg/ml (˜5 mM), and further diluted in neurobasal medium to final concentrations of 0.1 μM, 1 μM, and 10 μM. Lipoic acid was dissolved in EtOH at concentration 240 mM, and further diluted in the neurobasal medium to final concentrations of 10 μM, 25 μM, 50 μM and 100 μM. Vitamin E was dissolved in EtOH at a concentration of 100 mM, and further diluted in the neurobasal medium to final concentrations of 50 μM, 100 μM, 200 μM and 400 μM. Ginkgo biloba was dissolved in dH₂O at a concentration of 6 mg/ml, and further diluted in the neurobasal medium to final concentrations of 2.5 μ/ml, 5 μg/ml, 25 μg/ml, and 250 μg/ml. At the end of the treatment phase, the medium was replaced with 100-μl fresh, pre-warmed neurobasal medium plus B27 (-AO). The plates were returned to the incubator at 37° C. with 5% CO₂ for one hour. Subsequently, 20 μl MTS reagent was added to each well and the plates were incubated at 37° C. with 5% CO₂ for an additional two hours. The absorbance at 490 nm for each well was recorded with the BioRad plate reader (Model 550). Wells containing medium alone were used as blanks. Each data point is the average of three separate assay wells. Untreated cells were used as a control to calculate the cell viability and neuroprotective capacity. Two-week-old primary cultures were used for this set of study. See FIG. 1 for results.

Results

PAN-811 displayed good neuroprotective capacity at concentrations from 1-10 μM, even under harsh H₂O₂ treatment. Vitamin E and lipoic acid displayed minimal neuroprotective capacity under harsh treatment. Ginkgo biloba displayed a certain level of neuroprotection under harsh treatment.

PAN-811 displayed significant neuroprotection at 1-10 μM final concentration, even under harsh H₂O₂ treatment. The neuroprotective efficacy of PAN-811 significantly exceeded that of the other known neuroprotectants, Vitamin E, lipoic acid, and Ginkgo biloba.

### Example 2

### Effect of PAN-811 on Reactive Oxygen Species (ROS) Generation in Neuronal Cells

The purpose of this study was to assess the capability of PAN-811 to reduce ROS generation in a cell-based model of Alzheimer's disease-associated oxidative stress.

Materials used in this example are the same as in Example 1.

Primary cortical neurons were isolated from a 17-day-old rat embryonic brain and seeded in 96-well plates at 50,000 cells/well in regular neurobasal medium for 2-3 weeks. Twice, half the amount of medium was replaced with fresh neurobasal medium without antioxidants.

The primary cortical neurons were rinsed once with HBSS buffer and incubated with 10 μM 5-(and-6)-chloromethyl-2′,7′- dichlorodihydrofluorescein diacetate, acetyl ester (CM-H₂DCFDA) to pre-load the dye. The cells were then rinsed with HBSS buffer once and treated with PAN-811 at final concentrations of 0.1, 1, 5, and 10 μM for 1 hour, and further subjected to oxidative stress induced by hydrogen peroxide at 300 μM for 2 hours.

c-DCF fluorescence at 485/520 nm (Ex/Em) for each well was recorded with a BMG Polar Star plate reader and used to evaluate ROS generation in cells. Untreated cells loaded with the dye were used as controls to calculate the c-DCF fluorescence change. Each assay was performed in triplicate.

Results

The c-DCF fluorescence at 485/520 nm (Ex/Em) for each well was recorded with the BMG Polar Star plate reader. Wells containing cells without dye were used as blanks. Each data point is the average of three separate assay wells. Untreated cells loaded with the dye were used as a control to calculate the c-DCF fluorescence change. Two-week-old primary cultures were used for the study.

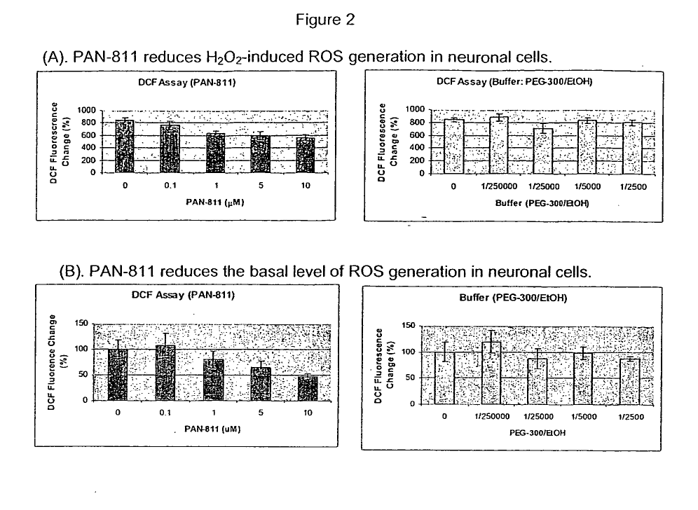

CM-H₂DCFDA is a cell-permeant indicator for reactive oxygen species (ROS), which is non-fluorescent until the acetate groups are removed by intracellular esterases and oxidation occurs within the cell. It has been widely employed to detect the generation of ROS in cells and animals. Here, it has been used as a tool to assess the effects of PAN-811 on ROS generation in neuronal cells following the procedures described in this example. As FIG. 2 illustrates, PAN-811 displayed good capacity to reduce H₂O₂-induced ROS generation, as well as basal level ROS generation in neuronal cells. The parallel control experiment using buffer, PGE-300/EtOH, instead of PAN-811, showed no effect on ROS generation in cells. Experiments were repeated four times in different batches of cells and similar results were obtained. See FIG. 2 for the representative experiment.

PAN-811 significantly reduced both H₂O₂-induced ROS generation (˜30% at 10 μM) and the basal level of ROS generation (˜50% at 10 μM) in primary neuronal cells.

Literature of Note

Gibson G E, Zhang H, Xu H, Park L C, Jeitner T M. (2001). Oxidative stress increases internal calcium stores and reduces a key mitochondrial enzyme. Biochim Biophys Acta. Mar 16;1586(2):177-89.

Chignell C F, Sik R H. (2003). A photochemical study of cells loaded with 2′,7′-dichlorofluorescin: implications for the detection of reactive oxygen species generated during UVA irradiation. Free Radic Biol Med. Apr 15;34(8):1029-34.

### Example 3

### PAN-811 is Neuroprotectant for Hypoxia- or Hypoxia/Hypoglycemia-Induced Neurotoxicity

The purpose of this example was to understand whether PAN-811 is able to protect hypoxia- or hypoxia/hypoglycemia (H/H)-induced neurotoxicity by examining its effects in vitro. As shown in the above examples, PAN-811 has been shown in related work to apply significant neuroprotection to primary neurons treated with H₂O₂.

The materials used in this example are the same as in Example 1. The LDH assay kit was obtained from Promega. (Abbreviations: BSS=balanced salt solution; CABG=coronary artery bypass graft; d.i.v.=days in vitro; EtOH=ethanol; H/H=hypoxia/hypoglycemia; LDH=lactate dehydrogenase; MCAO=middle cerebral artery occlusion; NB=neurobasal medium; NMDA=N-methyl-D-aspartate; PEG=polyethylene glycol)

Experiments were performed in a 96-well plate format. Cortical neurons were seeded at a density of 50,000 cells/well on a poly-D-lysine coated surface, and cultured in serum-free medium (NB plus B27 supplement) to obtain cultures highly enriched for neurons. Neurons were cultured for over 14 d.i.v. to increase cell susceptibility to excitatory amino acids (Jiang et al., 2001). Six replicate wells were treated as a group to facilitate assay quantitation.

As shown in Table 1 below, glucose concentration normally is over 2.2 mM in the brain. It decreases to 0.2 mM and 1.4 mM in the central core and penumbra, respectively, during ischemia. Glucose levels return to normal 1 or 2 hours after recirculation (Folbergroviá et al., 1995).

TABLE 1
Glucose Concentrations (mmol/kg)

| | | | 1-hour |
|---|---|---|---|
| | Sham | 2-hour MCAO | recirculation |
| Focus | 2.12 ± 0.18 | 0.21 ± 0.09 | 2.65 ± 0.19 |
| Penumbra | 2.20 ± 0.16 | 1.42 ± 0.34 | 2.69 ± 0.17 |

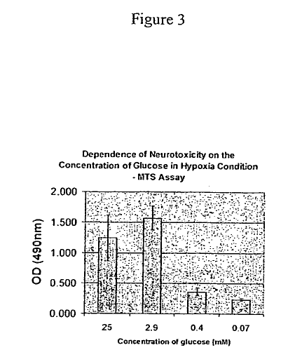

To understand the effect of glucose concentration on hypoxia-induced neurotoxicity, we tested different doses of glucose. As shown in FIG. 3, reduction of the glucose concentration to 2.9 mM did not result in neuronal cell death, by comparison to normal conditions where the glucose concentration is 25 mM. When glucose concentration went down to 0.4 mM, robust cell death occurred as indicated by the MTS assay.

To mimic the cerebral environments of a stroke, we established 3 in vitro model systems. The extreme H/H model (0.4 mM glucose) is a mimic of the environment in the central core of an infarct; the mild H/H model (1.63 mM glucose) is a mimic of the environment in the penumbra during MCAO; and the hypoxia only model (neurons in normal in vitro glucose concentration—25 mM) is a mimic of the environment in the penumbra after reperfusion since the possible cell death after reperfusion is predominantly a result of the hypoxic effect rather then energy failure.

Hypoxia/hypoglycemia was obtained by reducing glucose concentration down to 0.4 mM and 1.63 mM for extreme H/H and mild H/H, respectively. BSS (116.0 mM NaCl, 5.4 mM KCl, 0.8 mM MgSO4.e7H20, 1.0 mM NaH2PO4, 1.8 mM CaCl₂.2H₂O, 26.2 mM NaHCO₃, and 0.01 mM glycine) or BSS with 25 mM glucose were de-gassed for 5 minutes prior to use. Culture medium in the plates for hypoxia was replaced with BSS or BSS with glucose. Meanwhile, culture medium in the plates for normoxia was replaced with non de-gassed BSS or BSS with glucose. Cells were committed to hypoxic conditions by transferring the plates into a sealed container (Modular Incubator Chamber-101™, Billups-Rothenberg, Inc.), applying a vacuum for 20 minutes to remove oxygen or other gases from the culture medium, and then refilling the chamber with 5% CO₂ and 95% N₂ at a pressure of 30 psi for 1 minute. The level of O₂ in the chamber was determined to be zero with an O₂ indicator (FYRITE Gas Analyzer, Bacharach, Inc.). Culture plates were maintained in the chamber for 6 hours. As an experimental control, duplicate culture plates were maintained under normal culture condition (5% CO₂ and 95% ambient air) for the same duration. After a 6-hour treatment, plates were removed from the chamber and the medium in both the hypoxic and normoxic cultures was replaced with a termination solution (DMEM supplemented with 1×sodium pyruvate, 10.0 mM HEPES, and 1×N₂ supplement) containing 25 mM glucose and cultured in 5% CO₂ and 95% ambient air conditions. Neurons were treated with varying concentrations of PAN-811 or vehicle as a negative control. MK801 was utilized as a positive control. Mitochondrial function and cell death were evaluated at 24 or 48 hours post H/H insult with the MTS and LDH analyses (see below).

In the sole hypoxia model, the neurons were pre-treated with solvent or PAN-811 for 24 or 48 hours. Treatment with drug was continued during and subsequent to a 24-hour period of hypoxia. Cellular morphology and function (MTS and LDH assays) were measured 24 or 48 hours subsequent to the hypoxic insult.

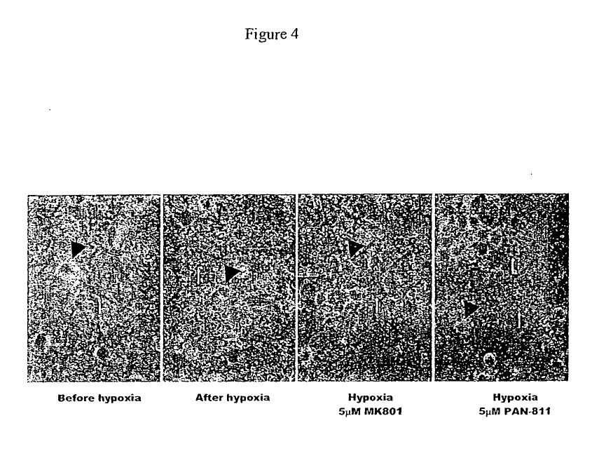

Neuronal cell death evaluated morphologically as seen in FIG. 4. Neurons prior to hypoxia are healthy with phase-brilliant cell soma (arrow head) and intact neuronal processes (open arrow). The processes and their branches form a dense network in the background. Hypoxia causes shrinkage of the cell body and collapse of the neuronal processes and network. PAN-811, as well as the glutamate NMDA receptor antagonist MK801 at doses of 5 μM, shows efficient protection from neuronal cell death and partial reservation of the neuronal processes.

The MTS assay is a colorimetric assay that measures the mitochondrial function in metabolically active cells. This measurement indirectly reflects cell viability. The MTS tetrazolium compound is reduced in metabolically active mitochondria into a colored formazan product that is soluble in tissue culture medium, and can be detected via its absorbance 490 nm. 20 μl of MTS reagent (Promega) are added to each well of the 96 well assay plates containing the samples in 100 μl of culture medium. The plate is then incubated in a humidified, 5% CO₂ atmosphere at 37° C. for 1-2 hours until the color is fully developed. The absorbance at 490 nm was recorded using a Bio-Rad 96 well plate reader.

The lactate dehydrogenase (LDH) assay is based on the reduction of NAD by the action of LDH. The resulting reduced NAD (NADH) is utilized in the stoichiometric conversion of a tetrazolium dye. If cell-free aliquots of medium from cultures given different treatments are assayed, then the amount of LDH activity can be used as an indicator of relative cell death as well as a function of membrane integrity. A 50 μl aliquot of culture medium from a well in tested 96-well plate is transferred into a well in unused plate and supplemented with 25 μl of equally-mixed Substrate, Enzyme and Dye Solutions (Sigma). The preparation is incubated at room temperature for 20-30 minutes, and then measured spectrophotometrically at wavelength of 490 nm.

Results

Sole Hypoxia Model

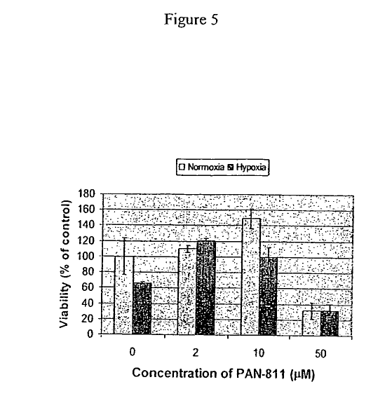

Cortical neurons were treated with PAN-811 for 48-hour prior to hypoxia; PAN-811 remained present during 24-hour hypoxia and for a 48-hour period subsequent to hypoxia. PAN-811 at dose of 21 μM completely blocked the cell death but 50 μM was toxic (see FIG. 5).

Cortical neurons were treated with 2 μM PAN-811, 1:80 green tea or 5 μM MK801 for 24 hours prior to, during and subsequent to a 24-hour period of hypoxia. PAN-811 demonstrated highest efficacy among reagents tested, completely blocking neuronal cell death and mitochondrial dysfunction.

Mild H/H Model

PAN-811 protected neurons from mild H/H- induced neurotoxicity before and during insult.

Embryonic (E17) rat cortical neurons were cultured for 15 days, treated with PAN-811 and vehicle 24-hours before and during hypoxia/hypoglycemia (6-hours). MTS and LDH assays were carried out 17 hours post to the insults. PAN-811 at 5 μM, but not a 1:1,520 dilution of PEG:EtOH (which corresponds to the mount of vehicle in 5 μM PAN-811), completely protected hypoxia/hypoglycemia-induced mitochondria dysfunction and neuronal cell death.

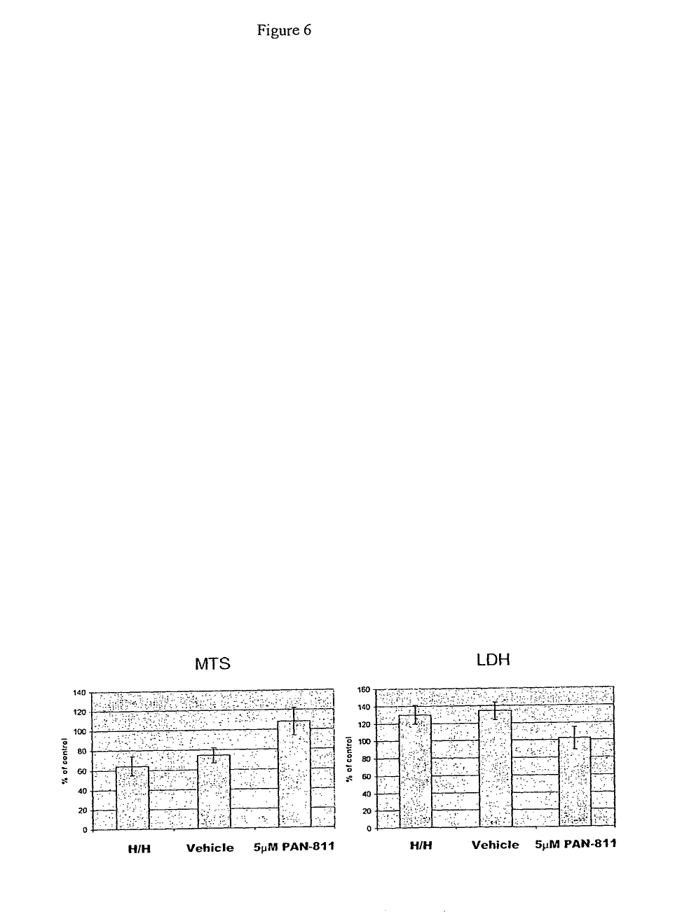

The data shown in FIG. 6 are representative. A summary of 6 experiments that cover a concentration range of 2-50 μM is shown in the following Table 2.

TABLE 2

| | Culture age | Pre- | | |
|---|---|---|---|---|
| | Comments | treatment | H/H duration | Post to H/H |
| Date | (days) | (hours) | (hours) | (hours) |
| Apr. 17, 2003 | 13 | 24 | 6 | 48 |
| | 2 μM: 100% protected | | | |
| May 2, 2003 | 22 | 24 | 6 | 24 |
| | 2 μM: 100% protected | | | |
| May 8, 2003 | 42 | 24 | 6 | 24 |
| | 2 μM: 100% protected | | | |
| Jul. 9, 2003 | 13 | 24 | 6 | 20 |
| | 2 μM: 100% protected | | | |
| Jul. 13, 2003 | 15 | 24 | 6 | 24 |
| | 10 μM: 100% protected | | | |
| Jul. 25, 2003 | 15 | 24 | 6 | 24 |

5 μM: 100% protected

** Test range started from 5 μMfor the experiments of Jul. 13, 2003 and Jul. 25, 2003

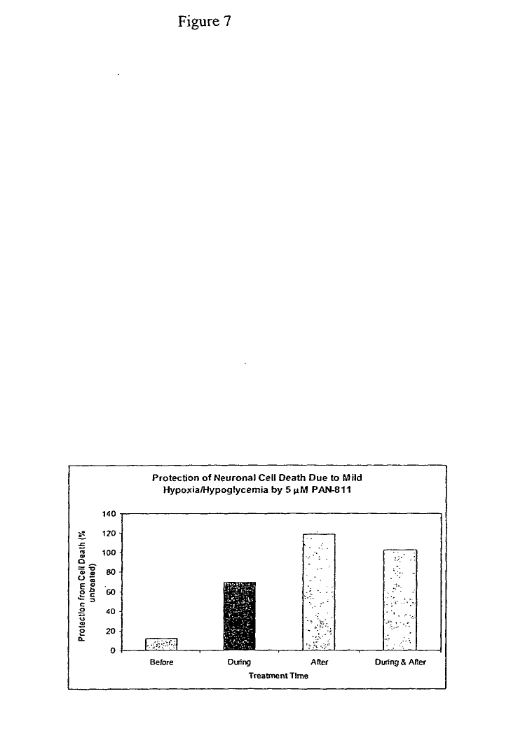

The neurons were cultured for 15 days, and treated with PAN-811 or PEG:EtOH (7:3) as vehicle for a 24-hour period prior to 6-hour H/H (Before Group). Alternatively the neurons were cultured for 16 days, and then treated with above reagents during 6-hour H/H (During Group), treated for a 6-hour H/H period and 48-hour period subsequent to the H/H (During and After Group), or treated for a 48-hour period subsequent to the H/H (After group). The LDH assay was carried out 48 hours after the period of H/H. The results demonstrated that PAN-811 protected neuronal cell death when treating the neurons during and especially after H/H, but marginally before H/H, see FIG. 7.

Extreme H/H Model

PAN-811 at ≦50 μM did not protect neuronal cell death (data not shown).

PAN-811 at 2 μM completely protected sole hypoxia- and mild H/H induced neurotoxicity. PAN-811 at 100 μM only partially blocked extreme H/H-induced neuronal cell death so PAN-811 is unlikely to be involved in energy metabolism.

PAN-811 significantly protects neurons from cell death when administered either during or subsequent to a hypoxic or ischemic insult.

The efficacy of PAN-811 is significantly greater than that of MK801 and/or green tea.

PAN-811 at 50 μM is toxic to neurons in long-term exposure (120-hour exposure).

Literature of Note

Jiang, Z. -G., Piggee, C. A., Heyes, M. P., Murphy, C. M., Quearry, B., Zheng, J., Gendelman, H. E., and Markey, S. P. Glutamate is a principal mediator of HIV-1-infected immune competent human macrophage neurotoxicity. J. Neuroimmunology 117(1 2):97-107, 2001.

Folbergrová, J., Zhao, Q., Katsura, K., and Siesjö, B. K. N-tert-butyl-phenylnitrone improves recovery of brain energy state in rats following transient focal ischemia. Proc. Natl. Acad. Sci. USA 92:5057-5061,1995.

### Example 4

### PAN-811 Displays Significant Neuroprotection in an In Vivo Model of Transient Focal Brain Ischemia

PAN-811 has shown significant neuroprotection in in vitro models of oxidative stress and ischemia. This work, coupled with the known toxicity profile and pharmacokinetic data on the compound, are highly compatible with its use in the treatment of stroke.

Materials are the same as those used in the above examples. In this example, MCAO is used as the abbreviation for middle cerebral artery occlusion.

Prior to embarking on in vivo studies, PAN-811 was tested in several cellular models of neurodegeneration.

Enriched neuronal cultures were prepared from 15-day-old Sprague-Dawley rat embryos. Using aseptic techniques, the rat embryos were removed from the uterus and placed in sterile neuronal culture medium. Using a dissecting microscope, the brain tissue was removed from each embryo, with care taken to discard the meninges and blood vessels. The cerebellum was separated by gross dissection under the microscope, and only cerebellar tissue was used for the culture. Cells were dissociated by trituration of the tissue and were plated at a density of 5×10⁵ cells/well onto 48-well culture plates precoated with poly(L-lysine). Cultures were maintained in a medium containing equal parts of Eagle's basal medium (without glutamine) and Ham's F-12k medium supplemented with 10% heat-inactivated horse serum, 10% fetal bovine serum, 600 μg/ml glucose, 100 μg/ml glutamine, 50 U/ml penicillin, and 50 μg/ml streptomycin. After 48 h, 10 μM cytosine arabinoside was added to inhibit non-neuronal cell division. Cells were used in experiments after 7 days in culture.

Cells were treated with varying amounts of PAN-811 (0-100 μM) for 24 hrs. Cell viability was determined in the MTT assay.

Four in vitro models of excitotoxicity were studied. Cells were either exposed to H/H conditions for 3 hrs or treated for 45 min with one of glutamate (100 μM), staurosporine (1 μM) or veratridine (10 μM). All cells were co-treated with or without PAN-811 (10 μM) in Locke's solution. At the conclusion of the respective excitotoxic exposures, the condition medium (original) was replaced. H/H was induced by incubating the cells in a humidified airtight chamber saturated with 95% nitrogen, 5% CO2 gas for 3 hrs in Locke's solution without added glucose.

Twenty-four hours after the excitotoxic insult, cell viability assessments were made. Cell damage was quantitatively assessed using a tetrazolium salt colorimetric assay with 3-(4,5-dimethylthiazol-2-yl)-2,5-diphenyltetrazolium (MTT; Sigma Chemical Co., St. Louis, Mo.). Briefly, the dye was added to each well (final concentration, 1.5 mg/ml), cells were incubated with MTT-acidified isopropanol (0.1 N HCl in isopropanol), and the absorbance intensity (540 nm) of each sample was measured in a 96-well plate reader. Values are expressed relative to vehicle-treated control cells that were maintained on each plate, and the percentage change in cell viability was calculated.

In Vivo Studies

Thirty-six male Sprague-Dawley rats (270-330 g; Charles River Labs, Raleigh, Va.) were used in this study. Anesthesia was induced by 5% halothane and maintained at 2% halothane delivered in oxygen. Body temperature was maintained normothermic (37±1° C.) throughout all surgical procedures by means of a homeothermic heating system (Harvard Apparatus, South Natick, Mass.). Food and water were provided ad libitum before and after surgery, and the animals were individually housed under a 12-h light/dark cycle. Rats were anesthetized and prepared for temporary focal ischemia using the filament method of middle cerebral artery occlusion (MCAO) and reperfusion. Briefly, the right external carotid artery was isolated and its branches were coagulated. A 3-0 uncoated monofilament nylon suture with a rounded tip was introduced into the internal carotid artery via the external carotid artery and advanced (approximately 22 mm from the carotid bifurcation) until a slight resistance was observed, thus occluding the origin of the MCA. The endovascular suture remained in place for 2 h and then was retracted to allow reperfusion of blood to the MCA. After MCAO surgery, animals were placed in recovery cages with ambient temperature maintained at 22° C. During the 2-h ischemia period and the initial 6-h post-ischemia period, 75-W warming lamps were also positioned directly over the top of each cage to maintain body temperature normothermic throughout the experiment.

The rats were treated 10 minutes prior to MCAO with 1/mg/kg PAN-811 via IV injection. PAN-811 was prepared as a stock solution in 70% PEG300, 30% EtOH. This stock was diluted 5-fold in sterile saline prior to injection (final concentration 1 mg/ml).

For each rat brain, analysis of ischemic cerebral damage was measured as a function of total infarct volume. This was achieved using 2,3,5-triphenyl tetrazolium chloride (TTC) staining from seven coronal sections (2-mm thick). Brain sections were taken from the region beginning 1 mm from the frontal pole and ending just rostral to the corticocerebellar junction. Computer-assisted image analysis was used to calculate infarct volumes. Briefly, the posterior surface of each TTC-stained forebrain section was digitally imaged (Loats Associates, Westminster, Md.) and quantified for areas (in square millimeters) of ischemic damage.

Results

In Vitro Studies

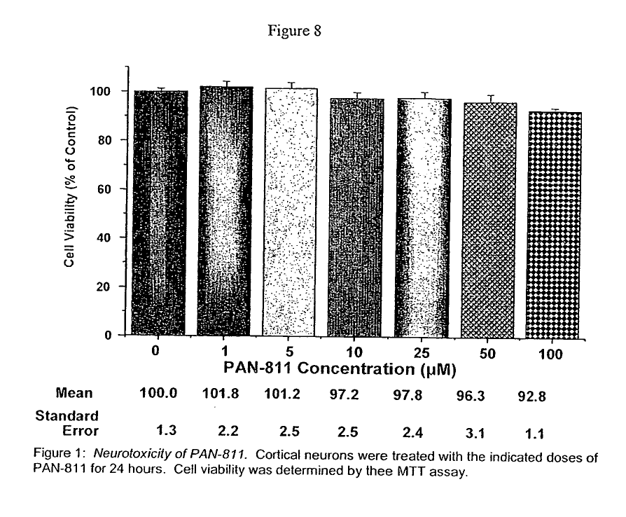

Neurotoxicity of PAN-811. Results are presented in FIG. 1. Essentially, PAN-811 showed only slight toxicity at concentrations up to 100 μM. Maximal toxicity was only 7.8% at the highest concentration tested (see FIG. 8).

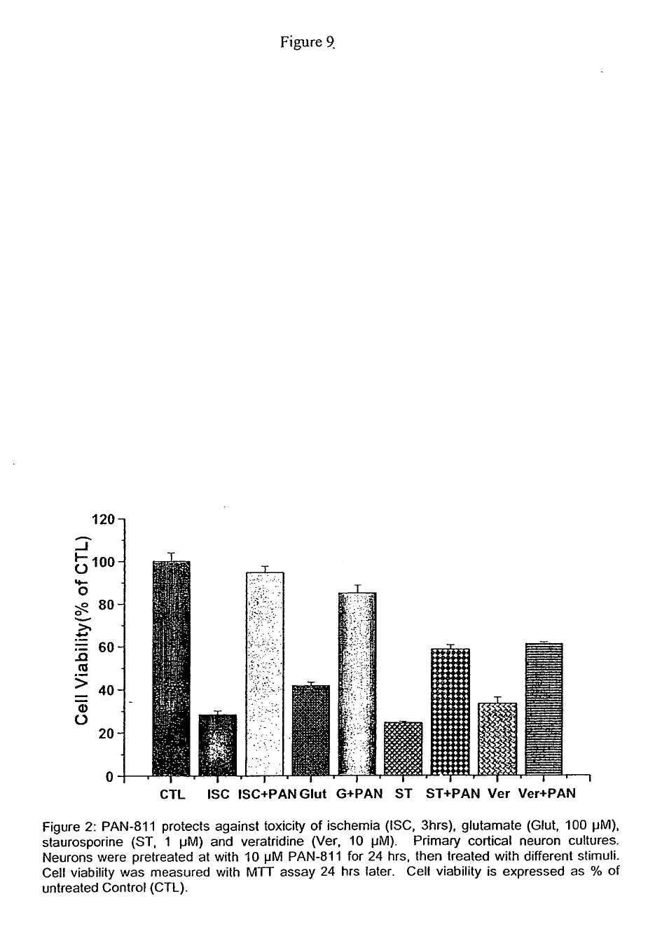

Neuroprotection due to PAN-811. PAN-811 was found to significantly protect neurons from for different excitotoxic insults (FIG. 2). Pre-treatment of neurons with 10 μM PAN-811 protected cells from the damage induced by a 3-hour period of hypoxia/hypoglycemia (92% protection), from 100 μM glutamate (˜75%), 1 μM staurosporine, an inhibitor of protein kinase C and inducer of apoptosis (˜47%) and 10 μM veratridine a sodium channel blocker (˜39%). See FIG. 9.

In Vivo Studies.

Results of this experiment are presented in Table 3. In total, 36 rats were used for the experiment, however 11 rats were excluded due to the following reasons: 4 rats died of severe stroke without complications of hemorrhage, 4 rats were excluded due to sub acute hemorrhage (3 of them died ≦24 h), 1 rat was excluded due to a fire drill during surgery, 1 rat was excluded due to being statistical outlier, and 1 rat died of overdose of halothane. Of the 7 rats that died (4 from severe strokes without SAH, and 3 with SAH), 6 were untreated (vehicle) rats and only 1 was treated with PAN-811. Vehicle treated rats had a mean infarct volume of 292.96 mm³ with a range from 198.75-355.81. PAN-811 treated rats had a mean infarct volume of 225.85 mm³ with a range 42.36-387.08. This represents a neuroprotection of 23% (p<0.05). For reasons yet to be determined, more severe injury was noted in the control group than is normally measured. Accordingly, the infarct size for the PAN-811 treated animals is also larger than expected for significant neuroprotection. Despite this issue the variability in both treatment groups was excellent (10% or less of the SEM) and was as good, if not better, than most of our previously published studies. PAN-811 is well tolerated and relatively non-toxic in both the in vitro and in vivo model systems. Pre-treated of neurons with 10 μM PAN-811 gave significant protection against for excitotoxic insults that result in neurodegeneration. Pre-treatment of rats 10 minutes prior to a period of transient focal brain ischemia with a single dose of PAN-811 (1 mg/kg) yielded a 23% reduction in average infarct volume.

Literature of Note

Williams A J, Dave J R, Phillips J B, Lin Y, McCabe R T, and Tortella F C. (2000) Neuroprotective efficacy and therapeutic window of the high-affinity N-methyl-D-aspartate antagonist conantokin-G: in vitro (primary cerebellar neurons) and in vivo (rat model of transient focal brain ischemia) studies. J Pharmacol Exp Ther. Jul;294(1):378-86.

TABLE 3
Table 3: Infarct Volume in mm³ of vehicle and PAN-811 treated rats.
Rats were treated with 1 mg/kg PAN-811 10 minutes prior to MCAO.
Infarct volume was determined 24 hours after surgery.

| | |
|---|---|
| Vehicle | |
| Treated | PAN-811 |

| | Infarct | | Infarct |
|---|---|---|---|
| Rat # | Volume | Rat # | Volume |
| R28 | 198.75 | R21 | 42.36 |
| R17 | 208.03 | R1 | 126.42 |
| R2 | 267.38 | R30 | 143.74 |
| R11 | 270.89 | R24 | 158.83 |
| R34 | 282.51 | R3 | 196.18 |
| R19 | 308.19 | R26 | 200.08 |
| R27 | 308.45 | R23 | 218.54 |
| R36 | 334.81 | R20 | 221.46 |
| R10 | 339.85 | R25 | 224.32 |
| R4 | 347.89 | R31 | 255.36 |
| R32 | 355.81 | R5 | 267.40 |
| | | R13 | 344.47 |
| | | R16 | 375.59 |
| | | R8 | 387.08 |
| Mean | 292.96 | Mean | 225.85 |
| SD | 53.60 | SD | 96.67 |
| SEM | 16.16 | SEM | 25.84 |
| N | 11 | n | 14 |

p value 0.05
% protection 23%

### Example 5

### Protection of Neurons from H₂O₂-induced Oxidative Stress by PAN-811

The purpose of this study was to assess the efficacy of PAN-811 as a neuroprotectant in a cell-based model of Alzheimer's disease-associated oxidative stress. Neuroprotection and cellular toxicity are determined. Various solvents were tested to determine their appropriateness as vehicles for the delivery of PAN-811.

The materials are the same as in the other examples.

Primary cortical neurons were isolated from a 17-day-old rat embryonic brain and seeded on 96-well plate at 50,000 cells/well in regular neurobasal medium for 2-3 week. Twice, half amount of medium was replaced with fresh neurobasal medium containing no antioxidants.

PAN-811 was dissolved in either EtOH or DMSO at 1 mg/ml (˜5 mM), in PEG-300/EtOH (70%/30%) at 5 mg/ml (˜25 mM), and further diluted in medium to final concentration at 1 μM, 5 μM, 20 μM and 50 μM. Neurons were pre-treated with PAN-811 or vehicle for 24 hours, and then subjected to oxidative stress induced by hydrogen peroxide (final concentration 60-70 μM). Controls include untreated cells (no PAN-811 and hydrogen peroxide treatment), cells treated with PAN-811 only, and cells exposed to hydrogen peroxide but not PAN-811. Untreated cells were used as a control to evaluate both toxicity and improved viability of neurons. Each assay was performed in triplicate. Equal volume of solvents (EtOH, DMSO, and PEG-300/EtOH) was added to cells to test the solvent effects on the assay.

After 24 hours, the cultures were evaluated for viability and mitochondrial function using a standard MTS Assay (Promega). The manufacturer's protocols were followed.

Results

Experiment 1

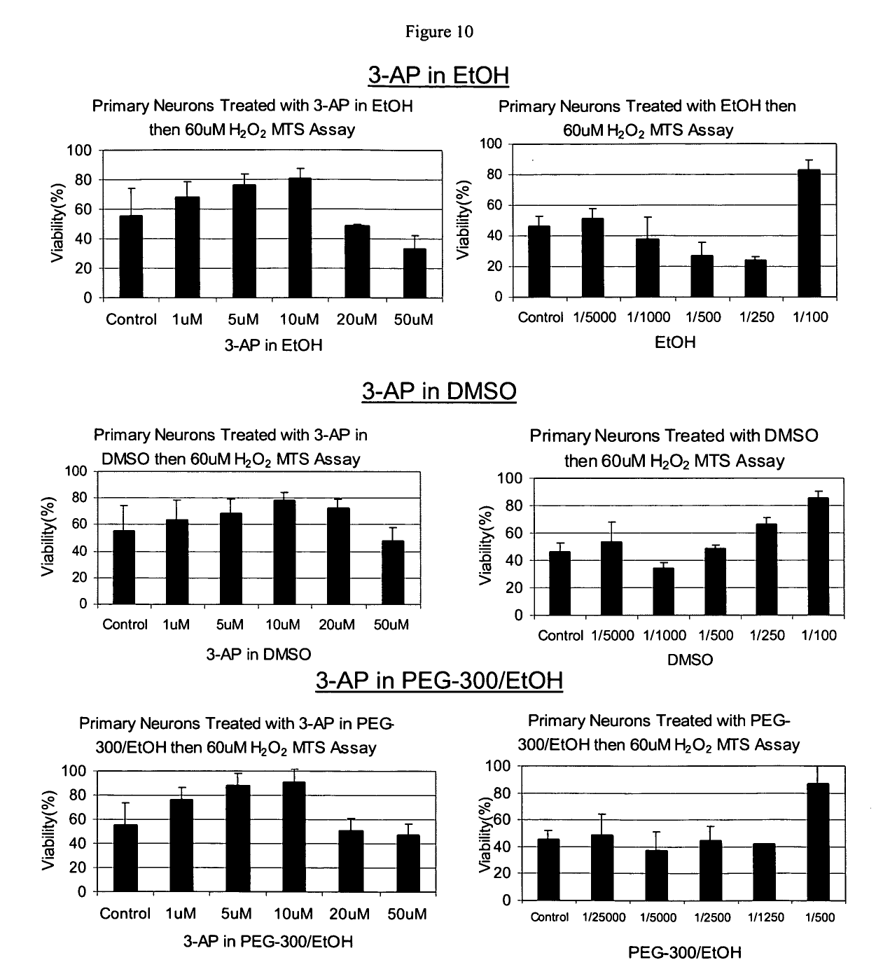

At the end of the treatment, all media were replaced with 100 μl fresh pre-warmed neurobasal medium plus B27 (-AO). The plates were put back into the incubator at 37° C. with 5% CO₂ for one hour, then 20 μl MTS reagent was added to each well and plates were incubated at 37° C. with 5% CO₂ for an additional two hours. The absorbance at 490 nm for each well was recorded with the BioRad plate reader (Model 550). Wells containing media alone were used as blanks. Each data point is the average of three separate assay wells. Untreated cells were used as a control to calculate the cell viability and neuroprotective capacity. Three-week-old primary cultures were used for this set of study. See FIG. 10 for results.

Experiment 2

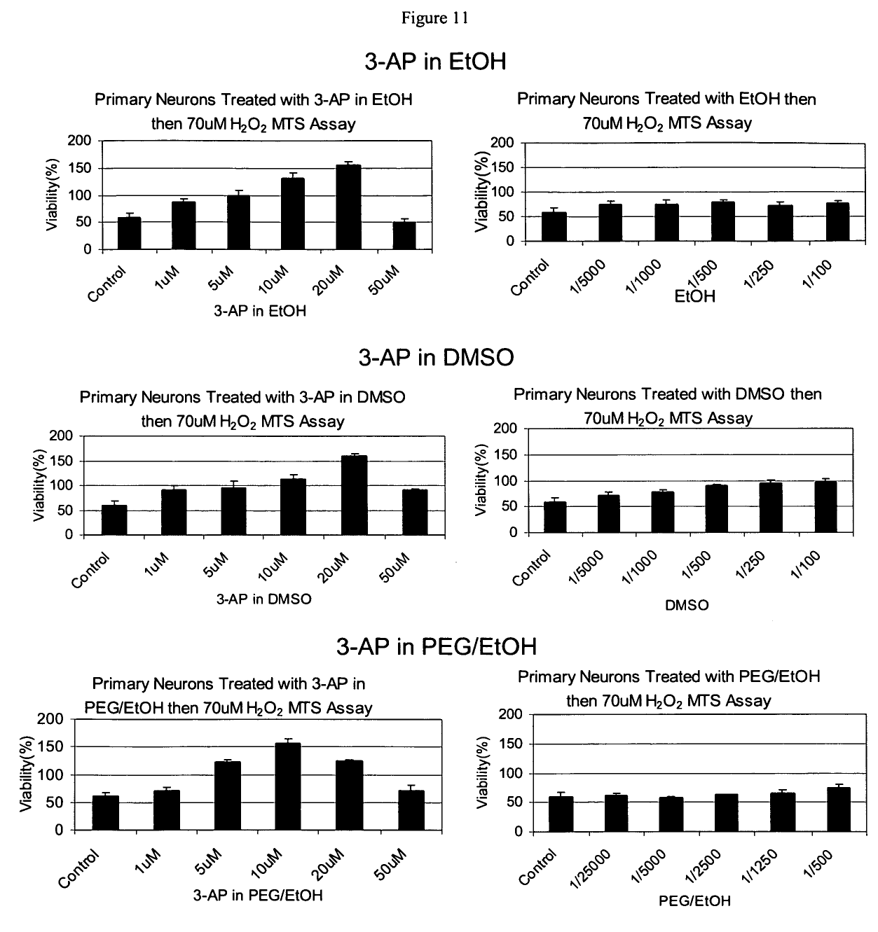

Experiments were carried out following the same procedures as experiment 1. Two-week-old primary cultures were used for this study. See FIG. 11 for results.

In these experiments, all three solvents showed minimal effects on the assay system at dilutions corresponding to final PAN-811 concentrations from 1-10 μM. DMSO displayed a certain level of neuroprotection at dilutions corresponding to final PAN-811 concentrations at or above 20 μM. EtOH and PEG-300/EtOH showed a certain level neuroprotection capacity at the dilution corresponding to a 50 μM final concentration of PAN-811. PAN-811 showed good neuroprotective capacity at 1-10 μM. PAN-811 has better solubility in PEG-300/EtOH comparing to EtOH alone.

PAN-811 showed good neuroprotective capacity at 1-10 μM final concentration. PEG-300 /EtOH showed very minimal interference with the assay system at dilutions corresponding to 1-20 μM of PAN-811, and is thus the best solvent for PAN-811 among the three solvents tested.

### Example 6

### PAN-811's Effects on Aβ1-42 or Aβ25-35 Induced Neurotoxicities

The purpose of this study was to show that PAN-811 is able to protect amyloid beta-induced neurotoxicity in vitro.

The following experiments utilized the same reagents in the above Examples, as well as amyloid beta peptide (Aβ1-42) from Sigma, Aβ25-35 from Oncogene or Bachem Bioscience Inc., and Aβ35-25 (reverse sequence) from Bachem Bioscience Inc. In addition to those already mentioned previously, other abbreviations used in this study are: AO=antioxidants; DPPH=diphenylpicrylhydrazyl; and Aβ=amyloid beta.

### Overall Study Design

Neuronal Culture

Experiments were performed in a 96-well plate format. Cortical neurons were seeded at a density of 50,000 cells/well on poly-D-lysine coated surface, and cultured in serum-free medium (NB plus B27 supplement containing AO or without AO) to obtain cultures high enriched for neurons. Neurons were cultured for over 14 d.i.v. to increase cell susceptibility to excitatory amino acids (Jiang et al., 2001, supra). Three to six replicate wells were treated as a group to facilitate assay quantitation.

Induction of Neurotoxicity-In Vitro Models

Lyophilized Aβs were dissolved in de-ionized and distilled water to a final concentration of 400 μM, and aliquoted and stored at −20° C. Before use, an aliquot of Aβ was removed from freezer and incubated at 37° C. for 48 hours. Neurons were cultured in NB with B27 supplement containing AO or without AO under 5% CO2 and 95% ambient air, 37° C. At 2 or 3 weeks, the neurons were treated with pre-incubated Aβ and either non-incubated Aβ or Aβ35-25 served as experimental controls.

Treatment with 2μM PAN-811 or 1:12,500 PEG:EtOH (7:3 as vehicle control) was started by at most 3 hours following Aβ insult. The neurons remained in culture post-treatment for different time periods, from 1 to 16 days.

Morphology Monitoring

Neuronal cell death was morphologically evaluated. Neurons prior to Aβ insult are healthy with phase-brilliant cell soma and intact neuronal processes. The processes and their branches form a dense network in the background. Aβ insult causes shrinkage of the cell body and collapse of the neuronal processes and network. PAN-811 at doses of 2μM shows efficient protection from neuronal cell death and reservation of the neuronal processes.

MTS Assay

The MTS assay is a colorimetric assay that measures the mitochondrial function in metabolically active cells. This measurement indirectly reflects cell viability. The MTS tetrazolium compound is reduced in metabolically active mitochondria into a colored formazan product that is soluble in tissue culture medium, and can be detected via its absorbance 490 nm. 10 μl of MTS reagent (Promega) are added to each well of the 96 well assay plates containing the samples in 50 μl of culture medium. The plate is then incubated in a humidified, 5% CO₂ atmosphere at 37° C. for 1 hour until the color is fully developed. The absorbance at 490 nm was recorded using a Bio-Rad 96 well plate reader.

LDH Assay

Lactate dehydrogenase (LDH) assay is based on the reduction of NAD by the action of LDH. The resulting reduced NAD (NADH) is utilized in the stoichiometric conversion of a tetrazolium dye. If cell-free aliquots of medium from cultures given different treatments are assayed, then the amount of LDH activity can be used as an indicator of relative cell death as well as a function of membrane integrity. A 35μl aliquot of culture medium from a well in a 96-well test plate is transferred into a well in unused plate and supplemented with 17.5 μl of equally mixed Substrate, Enzyme and Dye Solutions (Sigma). The preparation is incubated at room temperature for 30 minutes, and then measured spectrophotometrically at a wavelength of 490 nm.

### Experiment 1—Neurotoxicities of Aβ1-42 or Aβ25-35 Are ROS-Dependent

The purpose of this experiment was to find the optimal AO condition for Aβ1-42 or Aβ25-35 to induce neurotoxicity.

Primary neurons were isolated from cortex and striatum of 17-day-old rat embryonic brain and seeded on 96-well plates at 50,000 cells/well in regular neurobasal medium (Neurobasal Medium supplemented with B27-AO) for 2-3 weeks. Lyophilized Aβ1-42 or Aβ25-35 was dissolved in distilled, deionized water to a final concentration of 400 uM. Aliquots of the preparations were stored at −20° C. The day before experiment, the peptides were shifted from −20° C. to 37° C. and incubated overnight at 37° C.

The neurons at 12-16 d.i.v. were insulted by adding 2.5 ul per well of 400 uM Aβ1-42 or Aβ25-35 (final concentration: 10 uM). Untreated wells were taken as negative control and 25 uM glutamate treated wells were set as positive control. At the same time as or 3 hours after the initiation of Aβ insult, 1:5000 diluted PEG:EtOH (vehicle for PAN-811) was supplemented to the Aβ insulted wells. The neurons were continuously incubated at 37° C., 5% CO₂. The experiments were carried out in either 0% AO, 50% AO or 100% AO. Data were obtained from the readings of triplicate wells.

Results

Both Aβ1-42 and Aβ25-35 were shown to induce neurotoxicity under conditions of 0% AO.

Morphologically, neurons under normal conditions (i.e. without Aβ insult) show phase-brilliant cell bodies and neurites with branches forming the process network on the background. After insult with either Aβ1-42 or Aβ25-35 for a period of 16 days, shrinkage or collapse of neuronal cell bodies and interruption of neurites occurs with a loss of the process network.

LDH release from the cells indicates a cell membrane leakage and indirectly reflects neuronal cell death, which was used here for quantification of neurotoxicity of Aβ1-42 and Aβ25-35 and neuroprotection of PAN-811. Aβ1 -42 or Aβ25-35 by 16 day post-insult induces about a 40% increase in LDH assay of the culture medium in a reproducible manner.

Aβ25-35 is unable to induce neurotoxicity under the conditions of 50% and 100% AO. When neurons in a 100% or even a 50% AO environment were insulted with 10 μM of Aβ1-42 or Aβ25-35, no neuronal cell death could be observed. Under conditions of 100% AO, only 80 uM Aβ25-35 is able to induce neurotoxicity.

In view of the results obtained in this experiment, it is apparent that ROS must be present in the culture as a condition in order for Aβ1-42 or Aβ25-35 to induce neurotoxicity.

### Experiment 2—Neurotoxicity of Aβ25-35 Is Culture Age-Dependent

The purpose of this study was to find the optimal culture age of neurons for which Aβ1-42 or Aβ25-35 will induce neurotoxicity.

Primary neurons were isolated from cortex and striatum of 17-day-old rat embryonic brain and seeded on 96-well plates at 50,000 cells/well in regular neurobasal medium (Neurobasal Medium supplemented with B27-AO) for 2-3 weeks.

Lyophilized Aβ25-35 was dissolved in distilled, deionized water to a final concentration of 400 uM. Aliquots of the preparations were stored at −20° C. The day before the experiment, the peptides were shifted from −20° C. to 37° C. and incubated overnight at 37° C.

The neurons at 12-16 d.i.v., and at 21-23 d.i.v. were insulted by adding 2.5 ul per well of 400 uM Aβ25-35 (final concentration: 10 uM). Untreated wells were taken as negative control and 25uM glutamate treated wells were set as positive control. At the same time as or 3 hours after the initiation of Aβ insult, 1:5000 diluted PEG:EtOH (vehicle for PAN-811) was added to the Aβ insulted wells. The neurons were continuously incubated at 37° C., 5% CO₂. Data were generated from the readings of 3-6 replicate wells.

Results

A 12 -16 day culture period and 0% AO conditions are required for Aβ to induce neurotoxicity of younger neurons (12-16 d.i.v.). As mentioned above, neurons insulted with 10 uM of Aβ1-42 and Aβ25-35 need 12-16 days to show morphological changes and LDH release. The neurotoxicity of Aβ is ROS-dependent, since neuronal cell death under the presence of Aβ only occurred in 0% AO environment, but did not present under conditions of even 50% AO.

Only a 1-6 day period and 90% AO conditions are required for Aβ to induce neurotoxicity of older neurons (21-23 d.i.v.). Older neurons insulted with 10 uM Aβ25-35 only needed 1-6 days to exhibit neuronal cell death. The Aβ neurotoxicity occurred in 90% AO conditions, but not with 94% AO. Under Aβ insulted conditions, neurons loss the intact cell body and integrity of neurites with a 12-18% increase in LDH in the culture medium and a 7.5% decrease in the MTS readings.

Based on the results, it can be concluded that older neurons are more dependent on the concentration of AO in the culture medium and also become more susceptible to Aβ insult. Also, Aβ-induced neurotoxicity in older neurons is still ROS-dependent, since neuronal cell death only occurs under 90% AO, but not 94% AO, conditions.

### Experiment 3—Neurotoxicity of Aβ25-35 Is Specific

The purpose of this study was to determine whether or not Aβ25-35 induced neurotoxicity is specific by comparing it to a reversed sequence Aβ35-25 group as control.

Primary neurons were isolated from cortex and striatum of 17-day-old rat embryonic brain and seeded on 96-well plates at 50,000 cells/well in regular neurobasal medium (Neurobasal Medium supplemented with B27 -AO) for 2-3 weeks. Lyophilized Aβ25-35 and Aβ35-25 were dissolved in distilled, deionized water to a final concentration of 400 uM. Aliquots of the preparations were stored at −20° C. The day before experiment, the peptides were shifted from −20° C. to 37° C. and incubated overnight at 37° C.

The neurons at 21-23 d.i.v. were insulted by adding 2.5 ul per well of 400 uM Aβ25-35 (final concentration: 10 uM) or 2.5 ul per well of Aβ35-25 (final concentration: 10 uM). Untreated wells were used as negative control and 25 uM glutamate treated wells were set as positive control. At the same time as or 3 hours after the initiation of Aβ insults, 1:5000 diluted PEG:EtOH (vehicle for PAN-811) was added to the Aβ insulted wells. The neurons were continuously incubated at 37° C., 5% CO₂. Data were generated from the readings of 3-6 replicate wells.

Results

In contrast to Aβ25-35, Aβ35-25 cannot induce neurotoxicity of older neurons (21-23 d.i.v.) in 90% AO conditions by 1-6 day period. Neurons treated with Aβ35-25 presented healthier than the untreated group, with higher cell density, brighter phase contrast of cell bodies and denser neurites. The LDH level for this group is similar to that of the untreated group.

Thus, Aβ-induced neurotoxicity is specific, since reversed sequence Aβ, Aβ35-25, cannot induce neuronal cell death and cell membrane leakage in the experiments.

### Experiment 4—PAN-811 Efficiently Inhibits Aβ1-42 or Aβ25-35 Induced Neurotoxicity of Younger and Older Neurons Under 0% and 90% AO Conditions

The purpose of this study was to determine whether or not PAN-811 could protect co-cultured cortical and striatal neurons (younger or older) from Aβ1-42 or Aβ25-35 induced neuronal cell death.

Primary neurons were isolated from cortex and striatum of 17-day-old rat embryonic brain and seeded on 96-well plates at 50,000 cells/well in regular neurobasal medium (Neurobasal Medium supplemented with B27-AO) for 2-3 weeks.

Lyophilized Aβ1-42 and Aβ25-35 were dissolved in distilled, deionized water to a final concentration of 400 uM. Aliquots of the preparations were stored at −20° C. The day before the experiment, the peptides were shifted from −20° C. to 37° C. and incubated overnight at 37° C.

The neurons at 12-16 d.i.v. and 21-23 d.i.v. were insulted by adding 2.5 ul per well of either 400 uM Aβ1-42 or 400 uM Aβ25-35 (final concentration: 10 uM). Untreated and Aβ35-25-treated wells were taken as negative controls and 25 uM glutamate treated wells were set as the positive control. At the same time as or 2-3 hours after the initiation of Aβ insults, 1:5000 diluted PEG:EtOH (vehicle for PAN-811) as well as PAN-811 at concentrations of 0.25, 05., 1 and 2 μM were added to the Aβ insulted wells. The neurons were continuously incubated at 37° C., 5% CO₂. Data were obtained from the readings of 3-6 replicate wells.

Results

PAN-811 at a concentration of 2 μM fully blocks Aβ1-42 or Aβ25-35 induced neurotoxicity to younger neurons under 0% AO conditions. Neurons were monitored under a phase contrast microscope daily. No neuronal cell death in the Aβ1-42 or Aβ25-35 insulted groups was seen by 13 days. However, strong neuronal cell death in these groups was observed by 16 days.

After an insult with either Aβ1-42 or Aβ25-35 for a period of 12-16 days, neuronal cell bodies are shrunken or collapsed and there was interruption of neurites with a loss of process network. PAN-811 at a concentration of 2 μM showed well preserved neuronal and neurite morphologies no matter whether it was administered at the same time as or 3 hours after the Aβ1-42 or Aβ25-35 insult.

LDH release from the cells indicates a cell membrane leakage and indirectly reflects neuronal cell death, which was used here for quantification of neurotoxicity of Aβ1-42 and Aβ25-35 and neuroprotection of PAN-811. Aβ25-35 by day 16 post-insult induces about a 40% increase in LDH in the culture medium. PAN-811 at 2 μM, when administered at the same time with or 2-3 hours post the initiation of Aβ1-42 or Aβ25-35 treatment, results in bringing the LDH reading down to a level lower than untreated (control) group, and thus PAN-811 fully protects the neurons from the Aβ insults. These results were reproducible in additional experiments.

PAN-811 at concentrations of from 0.25 to 2 μM fully blocked Aβ1-42 and Aβ25-35 induced neurotoxicity in older neurons under 90% AO conditions. Neurons were monitored under a phase contrast microscope daily. Strong neuronal cell death in those groups was observed by 1-6 days post-insult. By this stage, neuronal cell bodies are shrunken or collapsed, and there was interruption of neurites with a loss of process network in both the Aβ1-42 and Aβ25-35 insulted groups. PAN-811 at concentrations of 0.25-2 uM well preserved neuronal and neurite morphologies no matter whether it was administered at the same time as or 2-3 hour after the Aβ1-42 or Aβ25-35 insult.

LDH release from the cells indicates a cell membrane leakage and indirectly reflects neuronal cell death, which was used hereby for the quantification of neurotoxicity of Aβ25-35 and neuroprotection of PAN-811. Under Aβ insult conditions, Aβ25-35 by 1-6 days post-insult induces about a 12-18% increase in the medium LDH reading and a 7.5% decrease in the MTS reading. PAN-811 at concentrations of from 0.25 to 2 μM, whether administered at the same time as or 2-3 hours after the initiation of Aβ25-35 treatment brings the LDH reading down to a level equal to or lower than the untreated (control) group. PAN-811 at concentrations of from 0.25 to 1 μM, when administered at 3 hours after the initiation of Aβ25-35 insult brings the MTS reading up to control level (untreated group). These results indicate that PAN-811 manifests full protection from the insult. The results were reproducible in further experiments.

From these results, it can be concluded that PAN-811 is capable of blocking Aβ1-42 or Aβ25-35 induced neurotoxicities in both younger neurons under 0% AO conditions and in older neurons under 90% AO conditions. The full protection occurs even when the dose of PAN-811 is as low as 0.25 μM and is administered 3 hours after the initiation of Aβ25-35 insult.

### Overall Study (Experiments 1-4) Observations

Generally, Aβ-induced neurotoxicity is culture age- and AO concentration-dependent. If using 2-week old mixed neurons, neuronal cell death only occurred in under conditions of 0% +AO. However, cell death is a long process, and needs at least 12-16 days post-insult to be observed to a measurable extent. The induced neurotoxicity is well protected by 2 μM PAN-811. Further, culture conditions of 50% AO are not sufficient for 10 μM Aβ25-35 to induce cell death. With conditions of 100% AO, 80 μM Aβ25-35 was needed to cause neurotoxicity by day 6 post-insult.

If using 3-week old mixed neurons, neither Aβ1-42 nor Aβ25-35 can induce neuronal cell death under 100% AO conditions. However, when AO concentration is reduced to 90%, Aβ25-35 at a dose of 10 μM could cause strong neuronal cell death after 1 day, which is well protected by a post-treatment (2 or 3h later) with 2 μM PAN-811. This indicates that the toxicity of Aβ is ROS-dependent and age-dependent, and that the optimal conditions could be between 90-100% +AO.

If using 5 or 6 week old mixed neurons, 10 μM Aβ25-35 still cannot induce neurotoxicity at 100% +AO conditions by 1 day or even 13 day post-insult. This strongly indicates that toxicity of Aβ is ROS dependent.

If using 7-week-old mixed neurons, the cells died in 100% +AO conditions by 1 day post-insult. In this case, 2 μM PAN-811 well preserved the morphology of neurons, but caused a slight reduction in the MTS reading. If using 8-week-old mixed neurons, a 50% reduction in AO in itself could cause neuronal cell death.

Moreover, the experiments show that AβB1-42 or Aβ25-35 induced neurotoxicity is specific. Compared with untreated groups, Aβ1-42 and Aβ25-35 caused strong neuronal cell death, indicated by morphological loss of intact cell body and integrity of neurites and the increase of LDH released in the medium. In contrast, Aβ35-25, a reversed sequence peptide of Aβ25-35, could never cause cell damage.

Further, the experiments show that Aβ1-42 or Aβ25-35 induced neurotoxicity is mediated by ROS. Most importantly, PAN-811 at a low dose is able to protect neurons from Aβ1-42 or Aβ25-35 insult under any of the conditions described above.

Based upon the above evidence, it is clear that ROS actively mediates Aβ-induced neuronal cell death. Previous experiments have revealed that PAN-811 can suppress intramitochondrial ROS accumulation and directly scavenge the stable free radical, DPPH, as well as chelate intracellular free calcium. Together with the above experiments, whereby neurons are insulted with either Aβl1-42 or Aβ25-35 at different conditions of culture age and AO concentration, and resulting in the demonstration that 2 μM PAN-811 can fully block Aβ-induced neuronal cell death of 3-week old neurons in 90% +AO conditions or of 2-week old neurons in 0% AO conditions, it is evident that PAN-811 exhibits a protective effect on neurons against amyloid peptides found in patients with Alzheimer's disease. Moreover, the above dose study indicates that PAN-811 at a dose as low as 0.25 μM can fully preserve neuronal morphology and mitochondrial function. Thus, it is evident that PAN-811 can protect neurons in patients with Alzheimer's disease.

Those of skill in the art will readily appreciate that the present invention is well adapted to carry out the objects and obtain the ends and advantages mentioned, as well as those inherent therein, and would know that various modifications in methods and amounts can be made in practicing the present invention without departing from the spirit or scope of the invention. Such modifications and variations are considered by the inventors as encompassed within the spirit of the invention, which is further defined in the appended claims.

We claim:

1. A method of ameliorating the progression of Alzheimer's disease, comprising treating or preventing neuronal damage due cellular insult by amyloid-beta by administering to a subject in need thereof a therapeutically effective amount of a compound of Formula I, or a pharmaceutically acceptable salt or prodrug thereof: 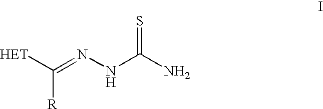
where HET is a 5 or 6 membered heteroaryl residue having 1 or 2 heteroatoms selected from N and S, and optionally substituted with an amino group; and R is H or C₁-C₄- alkyl,
whereby the compound provides protection of neurons from the affects of amyloid-beta.

2. The method of claim 1, wherein the compound, or a salt or prodrug thereof, administered to the subject is of Formula II: 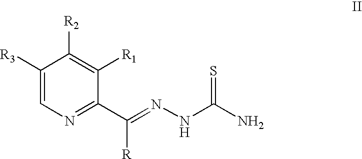
where R is H or C₁-C₄- alkyl; and R₁, R₂ and R₃ are independently selected from H and amino.

3. The method of claim 1, wherein the compound, or a salt or prodrug thereof, administered to the subject is of Formula III: 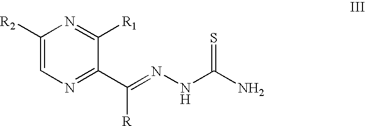
where R is H or C₁-C₄- alkyl; and R₁ and R₂ are independently selected from H and amino.

4. The method of claim 1, wherein the compound, or a salt or prodrug thereof, administered to the subject is of Formula IV: 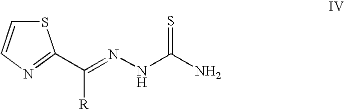
where R is H or C₁-C₄- alkyl.

5. The method of claim 1, wherein the compound, or a salt or prodrug thereof, administered to the subject is of Formula V: 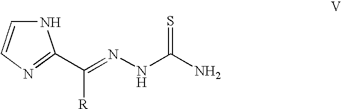
where R is H or C₁-C₄- alkyl.

6. The method of claim 1, wherein the compound, or a salt or prodrug thereof, administered to the subject is of Formula VI: 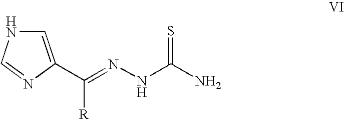
where R is H or C₁-C₄- alkyl.

7. The method of claim 1, wherein the compound, or a salt or prodrug thereof, administered to the subject is

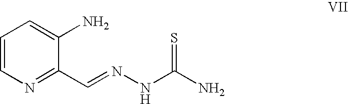

8. The method of claim 2, wherein R is methyl and R₁, R₂ and R₃ are H.

9. The method of claim 3, wherein R is methyl and R, and R₂ are H.

10. The method of claim 4, wherein R is methyl.

11. The method of claim 5, wherein R is H.

12. The method of claim 6, wherein R is H.
